(19) United States
(12) Patent Application Publication
(10) Pub. No.: US 2016/0022669 A1
(43) Pub. Date: Jan. 28, 2016

(54) SELF-SOLIDIFYING BARRIER IMPLANT AND METHOD OF MAKING THE IMPLANT

(71) Applicant: Steven A. Melman, Boca Raton, FL (US)

(72) Inventor: Steven A. Melman, Boca Raton, FL (US)

(21) Appl. No.: 14/804,501

(22) Filed: Jul. 21, 2015

Related U.S. Application Data
(60) Provisional application No. 62/027,342, filed on Jul. 22, 2014.

(51) Int. Cl. A61K 31/496; A61K 31/573; A61K 9/06; A61K 47/34

(52) U.S. Cl. CPC A61K 31/496; A61K 47/34; A61K 31/573; A61K 9/06

(57) ABSTRACT

Provided are bioerodible formulations that can be implanted into a cavity of a mammal as a liquid or semi-liquid and which solidifies upon exposure to body temperature of the mammal to form an implant. The implant erodes over a period of time and eluted a drug. The implant forms a seal with the surrounding tissue to prevent the entry of bacterial pathogens and enhance delivery of the Al at the desired foci.

This application claims priority to U.S. provisional patent application Ser. No. 62/027,342, filed 22 Jul. 2014, the complete disclosure of which is incorporated herein by reference.

### FIELD OF THE INVENTION

The present invention relates to implants that are formed in-situ in open cavities of mammalian tissue, including oral, nasal, rectal, vaginal, otic, periodontal pockets, surgical incisions, and open wounds. The invention relates particularly to bioerodible implants that are drug eluting, and that act as a barrier when implanted against infiltration by exogenous pathogens.

### BACKGROUND OF THE INVENTION

Biodegradable polymers are a large and growing segment of the medical device market, and have found varying applications for use. They are predominantly used in sutures, but have also found use in orthopedic fixation devices such as interference screws in the ankle, knee, and hand areas; as tacks and pins for ligament attachment and meniscal repair; as suture anchors; and as rods and pins for fracture fixation.

Poloxamers and their use have a long history. The structure consists of a hydrophobic central core of propylene oxide, flanked by hydrophilic ethylene oxide on both sides. Poloxamers are soluble in water and other polar and non-polar solvents and are regarded as chemically inert. Commercially, poloxamers are available from BASF as flakes (denoted by “F”), paste (denoted by “P”), liquid (denoted by “L”) and micronized (denoted by “micro”).

Poloxamers show temperature dependent thermoreversible properties. Poloxamer 407 (F127) is the most well studied poloxamer for this behavior. Generally, this behavior has been studied in 20-30% w/w aqueous solutions, which are liquid at low temperature (2-5° C.) and turn into gel at room temperature (22-25° C.). This gelation temperature is dependent on the molecular weight and the percentage of the hydrophobic portion, hence the gelling temperature decreases as both the molecular weight and the hydrophobic fraction increases. In general, the gelation temperature increases in the order of F127<F108<F 87<F68<F44. The gelation temperature can also be modulated by varying the percentage of F127, or mixing it with one or more other poloxamers. The three pharmaceutically relevant (due to availability and approved for use in pharmaceutical products) poloxamers are F127, F108, and F68.

U.S. Pat. No. 8,501,230 (Alur) discloses the use of poloxamers in combination with xanthan gum to form a drug-eluting bioerodible implant. The complete disclosure of this patent is incorporated herein by reference.

### SUMMARY OF THE INVENTION

The formulation exists at a viscosity that can be easily injected into a mammalian cavity, and, subsequently, remains in place for the liquid to solidify. The implants which result from the injection disintegrate in adjacent aqueous-based extracellular fluids at a predictable rate over an extended period of time, elute active ingredient(s) at a controlled rate, provide a barrier against entry of infectious pathogens, and are completely cleared and excreted from the body via normal pathways of elimination.

The formulation can be injected into any mammalian cavity, which includes but is not limited to nasal, oral, rectal, vaginal, dental sulci, or implanted at the site of a surgical incision or open wound before the incision or wound is sutured together. The implant preferably includes an active drug that is eluted to surrounding tissue, while at the same time acting as a barrier to prevent further entry of pathogens into the cavity. The method can be performed with a liquid formulation that provides the following advantages:

(1) The liquid formulation (liquid below a mammalian body temperature) converts to a solid or semi-solid gel in situ when injected into the cavity, penetrates into any crevices located within the cavity, and is heated to the mammalian body temperature;

(2) The liquid formulation contains an active drug, such as an antibiotic, that is eluted over time from the implant onto the surfaces inside the cavity, and that eliminates any infection from bacteria that might otherwise invade the cavity;

(3) The implant is bioadhesive, and seals the tissue together from within the cavity, to form a barrier against further bacterial invasion and to adhere to affected tissue to insure maximum delivery of the active ingredient (Al), for example, treating inflamed tissue using a corticosteroid; and

(4) The implant is bioderodible, so that it dissolves or disintegrates in adjacent extracellular fluids over time and is cleared from the implant site by normal elimination pathways.

### DETAILED DESCRIPTION OF THE INVENTION

### Definitions and Use of Terms

As used in this specification and in the claims, which follow, the singular forms “a,” “an” and “the” include plural referents unless the context clearly dictates otherwise. Thus, for example, reference to “an ingredient” includes mixtures of ingredients; reference to “an active pharmaceutical agent” includes more than one active pharmaceutical agent, and the like.

“Pharmaceutically acceptable” means that the compound or mixture is useful in preparing a pharmaceutical composition that is generally safe, non-toxic and neither biologically nor otherwise undesirable and includes that which is acceptable for human pharmaceutical use.

“Veterinary acceptable” means that the compound or mixture is useful in preparing a composition that is generally safe, non-toxic and neither biologically nor otherwise undesirable and includes that which is acceptable for veterinary use.

“Therapeutically effective amount” means that amount which, when administered to a mammal for treating or preventing a disease, is sufficient to effect such treatment or prevention for the disease.

The term “mammal” includes humans and warm blooded animals (veterinary applications). Mammals have body temperature of about 36° C. to about 41° C. “Mammalian cavity” includes, but is not limited, to cavities such as nasal, oral, rectal, vaginal, otic, dental sulci, surgical incision, or open wound.

When ranges are given by specifying the lower end of a range separately from the upper end of the range, it will be understood that the range can be defined by selectively combining any one of the lower end variables with any one of the upper end variables that is mathematically possible.

When used herein the term “about” or “ca.” will compensate for variability allowed for in the pharmaceutical industry and inherent in pharmaceutical products, such as differences in product strength due to manufacturing variation and time-induced product degradation. The term allows for any variation, which in the practice of pharmaceuticals would allow the product being evaluated to be considered bioequivalent in a mammal to the recited strength of a claimed product.

The liquid formulation preferably employs polymeric chemistry to self-solidify. Polymers for practicing the invention can be either natural or synthetic. In general, synthetic polymers offer greater advantages than natural materials because they can be tailored to give a wider range of properties and more predictable lot-to-lot uniformity than can materials from natural sources. Synthetic polymers also represent a more reliable source of raw materials, one free from concerns of immunogenicity.

The general process for selecting a polymer for use as a biomaterial is to match the mechanical properties and the time of degradation to the needs of the application. This invention also imposes three additional demands on the polymer selected: the polymer must self-harden to a solid or semi-solid state when implanted into a body cavity; the polymer should elute a drug at a suitable rate of release; and the polymer must form a barrier against entry of pathogens into the cavity.

Factors that affect the mechanical performance of biodegradable polymers are well known to the polymer scientist, and include monomer selection, initiator selection, processing conditions, and the presence of additives. These factors in turn influence the polymer's hydrophilicity, crystallinity, melt and glass-transition temperatures, molecular weight, molecular-weight distribution, end groups, sequence distribution (random versus block), and presence of residual monomer or additives.

Biodegradation can be accomplished by synthesizing polymers that have hydrolytically unstable linkages in the backbone. The most common chemical functional groups with this characteristic are esters, anhydrides, orthoesters, and amides.

Technology for making the biodegradable implant generally falls into one of two categories: thermo-gelling (sol to gel) polymers that convert from liquid to solid or semi-solid when heated to body temperature, and solvent eluting polymeric compositions that solidify as solvent elutes from the composition into the body and the polymer becomes more concentrated.

The formulation is a liquid at a temperature below a mammalian body temperature, such as common refrigeration temperatures, and a solid or gel at a mammalian body temperature. A preferred thermo-gelling composition comprises a poloxamer. The term “poloxamer” refers to any of the group of polyoxyethylene-polyoxypropylene block copolymers known in the art. Poloxamers are also known by the trade name Pluronics, and are nonionic block copolymers composed of a central hydrophobic chain of polyoxypropylene (polypropylene oxide) flanked by two hydrophilic chains of polyoxyethylene (polyethylene oxide). Because the lengths of the polymer blocks can be customized, many different poloxamers exist that have slightly different properties. These polymers are commonly named with the word Poloxamer followed by a number to indicate which polymer is being discussed (e.g. Poloxamer 407). The poloxamer can optionally be modified with, for example, a chain extender such as terephthaloyl chloride, to improve the stability of the gel and decrease its rate of degradation in vivo. See Ahn et al., POLYM. INT. 54:842-847 (2005). Different types of poloxamers can also be mixed to vary the properties of the material. Preferred poloxamers are F127, F108, and F68, with F127 being most preferred.

The formulation can comprise any desired amount of the poloxamer, for example from 10 to 25% by weight of the poloxamer, and more preferably from 15 to 20% by weight of the poloxamer based on the total weight of the formulation. When F127 is utilized in the formulation, the amount of poloxamer is preferably from 17 to 20% by weight. The formulation also comprises water, and in one embodiment the composition comprises 70 to 90 weight parts water, and in a preferred embodiment the formulation comprises from 75 to 85 weight parts water.

The formulation must be in form of solid or gel at a mammalian body temperature. For example, the formulation can be a gel at a temperature between room temperature and a mammalian body temperature, and in the form of an injectable liquid at temperatures below room temperature. In a particularly preferred embodiment, the implant is completely bioerodible, meaning that the entire formulation, once it has dissolved or disintegrated, can be processed via normal elimination pathways and excreted from the human body, optionally by the kidneys in the case of poloxamers.

The liquid form of the formulation in one embodiment can have a viscosity in the liquid form of from 100,000 to 1,000,000 cps, before being implanted and solidifying. In another embodiment, the viscosity of the liquid form of the formulation ranges from 200,000 to 500,000 cps.

The dilution of the polymer in a solvent can have an impact on the properties of the poloxamer, including the temperature of gel conversion and the extent of conversion at a given temperature, which effects are well known in the art.

Hydrophobic polymers that degrade by surface erosion rather than by bulk hydrolytic degradation can also be used in the process of the invention, especially when using drugs that are hydrolytically unstable. Two classes of these polymers are the polyanhydrides and the polyorthoesters. Polyanhydrides can be synthesized via the dehydration of diacid molecules by melt polycondensation, and their degradation times can be adjusted from days to years according to the degree of hydrophobicity of the monomer selected. Polyorthoesters also degrade by surface erosion, and degradation rates can be controlled by incorporation of acidic or basic excipients.

Any suitable biocompatible organic solvent can be employed to liquefy the composition, provided the biocompatible organic solvent is miscible to dispersible in aqueous medium or body fluid and can effectively dissolve the thermoplastic polyester. Suitable biocompatible organic solvents include, for example, N-methyl-2-pyrrolidone, 2-pyrrolidone, 2-ethoxyethanol, 2 ethoxyethyl acetate, ethyl acetate, ethyl lactate, ethyl butyrate, diethyl malonate, diethyl glutarate, tributyl citrate, acetyl-tri-n-hexylcitrate, diethyl succinate, 1 tributyrin, isopropyl myristate, propylene carbonate, dimethyl carbonate, ethylene glycol dimethyl ether, propylene glycol, 1,3-butylene glycol, caprolactone, N-dimethylformamide, dimethylacetamide, dimethyl sulfoxide, dimethyl sulfone, caprolactain, decylmethylsulfoxide, oleic acid, N,N-dimethyl-m-toluamide, 2,2 dimethyl-1,3-dioxolane-4-methanol, triacetin, ethyl acetate, benzyl alcohol, benzyl benzoate, solketal, glycofurol, dodecylazacycloheptan-2-one, glycerol, glycerin, dimethyl sulfoxide (DMSO) or any combination thereof.

A suitable biocompatible organic solvent that can diffuse into body fluid so that the liquid implant coagulates or solidifies can be used. Preferably, the biocompatible organic solvent for the biodegradable polymer be non-toxic and otherwise biocompatible.

The solubility of the biodegradable thermoplastic polyesters in the various biocompatible organic solvents may differ depending upon their crystallinity, their hydrophilicity, hydrogen-bonding, and molecular weight. Thus, not all of the biodegradable thermoplastic polyesters will be soluble in the same biocompatible organic solvent, but each biodegradable thermoplastic polymer or copolymer should have its appropriate biocompatible organic solvent. Lower molecular-weight polymers will normally dissolve more readily in the solvents than high molecular weight polymers. As a result, the concentration of a polymer dissolved in various solvents will differ depending upon type of polymer and its molecular weight. Conversely, the higher molecular-weight polymers will normally tend to coagulate or solidify faster than the very low-molecular-weight polymers. Moreover the higher molecular weight polymers will tend to give higher solution viscosities than the low molecular-weight materials.

Any type of drug can be incorporated in the biodegradable implant, including analgesics, local anesthetics, antimicrobials, antibacterials, anti-inflammatories, anti-infectives, antifungal, vasoconstrictors, hormones, or any other desired drug. Therapeutically effective amounts of these drugs can be released immediately after implanting and, for example, up to about 30 days after implanting. In a preferred embodiment, a therapeutically effective amount of the drug is released up to about 15 days following implantation. The type drug incorporated in the formulation and amount can depend upon whether the formulation is for human use or veterinary use.

Analgesic drugs that can be incorporated into the dosage form include, but are not limited to, acetaminophen, ibuprofen, methylsalicylate, menthol, camphor, methylnicotinate, triethanolamine salicylate, glycol salicylate, or salicylamine.

Local anesthetics that can be incorporated into the dosage form include, but are not limited to, lidocaine hydrochloride, oxybuprocaine hydrochloride, procaine, benzocaine, xylocaine, etidocaine, cocaine, benoxinate, dibucaine hydrochloride, dyclonine hydrochloride, naepaine, phenacaine hydrochloride, piperocaine, proparacaine hydrochloride, tetracaine hydrochloride, hexylcaine, bupivacaine, and mepivacaine.

Suitable antimicrobials include, but are not limited to, iodine, povidone iodine, benzalkonium chloride and chlorhexidine gluconate.

Suitable antibacterial drugs include, but are not limited to, the beta-lactam antibiotics, tetracyclines, chloramphenicol, clindamycin, neomycin, gramicidin, bacitracin, polymixin, sulfonamides, aminoglycoside antibiotics, tobramycin, nitrofurazone, nalidixic acid and analogs, aminoglycosides such as gentamycin and amikacin, fluoroquinolines such as baytril, and penicillins in their various forms, salts, bases, acetates, phosphates, sulfates, and aqueous, and derivatives thereof. The antibacterial drug can be added in any desired amount. Examples of suitable amounts of the antibacterial drug include from 0.01 to 10% by weight. All % by weight are based on the total weight of the composition unless otherwise stated. The drug can be added in amounts, for example, of 0.1 to 100 grams/ml, as desired.

Suitable anti-inflammatories include, but are not limited to corticosteroids such as cortisone, hydrocortisone, betamethasone, dexamethasone, fluocortolone, prednisolone, triamcinalone, indomethacine, hydrocortisone diethylaminoacetate, and sulindac in their various forms, salts, bases, acetates, phosphates, sulfates, and aqueous, and derivatives thereof. The anti-inflammatory drug can be added in any desired amount. Examples of suitable amounts of the anti-inflammatory drug include from 0.01 to 10% by weight. The anti-inflammatory can be added in amounts, for example, of 0.1 to 100 grams/ml.

A preferred type of anti-inflammatory includes the group of “Soft Steroids.” The soft drug approach can be utilized as a means of delivering steroids close to their site of action while reducing the degree of systemic exposure and thus limiting or eliminating the associated systemic and local side effects. The soft steroids can be delivered in a once daily formulation, once weekly, or less, as desired, to enhance patient compliance.

For example, the soft steroids can be used for allergies, ear diseases, or any disease where there might be chronic inflammatory issues, or any disease where inhibiting inflammation may reduce the trigger which causes clinical signs, such as allergies. If the inflammation is reduced, then the threshold for allergy may be increased to make it less likely for clinical signs to appear. In addition, if inflammation is reduced, then other allergens that the mammal may be less sensitive to but because of the inflammation they can cause clinical signs, which phenomenon is known as PRIMING. For example, a person with a grass allergy may be able to sleep on a feather pillow during none allergy season, but when grass pollen is in the air they cannot sleep on the feather pillow.

The present invention can be used to deliver steroids over a long term sustained period to prevent, alleviate, or otherwise treat diseases, such as for example allergies

Nonsteroidal anti-inflammatory drugs (NSAIDs) can be utilized as desired.

Suitable anti-infectives include, but are not limited to, bifonazole, siccanin, bisdequalinium acetate, clotrimazole, salicylic acid, sulfamethoxazole sodium, erythromycin and gentamicin sulfate.

Suitable antifungals include, but are not limited to, azoles such as miconazole, ketoconazole, fluconazole, itraconazole, amphotericin and other more potent antifungals. The antifungal drug can be added in any desired amount. Examples of suitable amounts of the antifungal drug include from 0.01 to 15% by weight. The antifungal can be added in amounts, for example, of 0.1 to 150 grams/ml.

A suitable vasoconstrictor is epinephrine, however, other vasoconstrictors can be utilized.

An example of a nasal formulation includes epinephrine as the active drug. The nasal formulation can be applied directly to an epistaxis case with the benefit being the occlusion of the gel and the delivery of the epinephrine to the focus of bleeding without the undesirable abrasiveness of gauze packing.

An example of a dental formulation includes an antibiotic, such as clindamycin or aminoglycoside, as the active drug. The dental formulation when gelled at the delivery site, such as in a cavity of an upper tooth, gum or pocket of infection, the gelled formulation will stay in place.

The formulations can be applied to any cavity on a mammal, such as otic, nasal, foot abscess, horses hoof, tonsil pocket, horses prepuce, eye, joints, vaginal, rectal (anal), and any other cavity.

An example of a preferred anti-fungal formulation comprises 17-20% by weight of poloxamer F127, 0.015% (0.15 mg/ml) by weight ketoconazole, 1% by weight hydrocortisone, and the balance water.

The anti-fungal formulation can be used to treat otic, nasal, vaginal, oral, dental, rectal, ophthalmic, dermal, digital yeast and fungal disorders.

The formulation can also contain one or more conventional pharmaceutical excipients selected from stabilizers, antioxidants, buffers and pH regulating agents. When used for veterinary purposes, the formulation can also contain one or more conventional veterinary suitable excipients selected from stabilizers, antioxidants, buffers and pH regulating agents. The formulation preferably excludes xanthan gum.

The formulation can contain synergists, pH buffers, and chelating agents, such as when using tromethamine and EDTA, which can enhance the effects of fluorquinolones and aminoglycosides. They can also have primary effects against bacteria and yeast. See U.S. Pat. No. 6,538,155 (Melman) the complete disclosure of which is incorporated herein by reference.

While the invention has been described with reference to particular embodiments, it will be understood by those skilled in the art that various changes may be made and equivalents may be substituted for elements thereof without departing from the scope of the invention. In addition, many modifications may be made to adapt a particular situation or material to the teachings of the invention without departing from the scope of the invention. Therefore, it is intended that the invention not be limited to the particular embodiments disclosed as the best mode contemplated for carrying out this invention, but that the invention will include all embodiments falling within the scope and spirit of the appended claims. In addition, the logic flows depicted in the figures do not require the particular order shown, or sequential order, to achieve desirable results.

1. A method of delivering an anti-fungal drug formulation to a bodily cavity in a mammal comprising:
providing formulation comprising a therapeutically effective amount of an antifungal drug dispersed or solubilized in a carrier, wherein the carrier comprises a poloxamer in an amount such that the formulation is the form of a gel at the mammal body temperature and in the form of a liquid at a temperature lower than the mammal body temperature; and
forming a barrier against pathogen entry into a cavity in the mammal by inserting the formulation into a cavity of the mammal, whereupon when the formulation becomes warmer by heat from the mammal the formulation gels to form an implant and adheres to tissue around a periphery of the cavity to form the barrier.

2. The method of claim 1, wherein the formulation comprises 17-20% by weight of poloxamer F127, 0.01-15% by weight antifungal drug.

3. The method of claim 1, wherein the formulation further comprises an anti-inflammatory drug.

4. The method of claim 3, wherein the anti-inflammatory drug is provided in an amount to treat, prevent or alleviate an allergy over a time period.

5. The method of claim 1, wherein the formulation comprises 17-20% by weight of poloxamer F127, 0.015% by weight ketoconazole, 1% by weight hydrocortisone, and the balance water.

6. The method of claim 1, further comprising eluting the antifungal drug from the gel over a desired time period.

7. The method of claim 1, further comprising eluting the antifungal drug from the gel at a desired rate.

8. The method of claim 1, further comprising adhering the gel to an affected tissue to deliver the antifungal agent directly to the affected tissue.

9. The method of claim 1, wherein the gel is biodegradable, and the method further comprising dissolving or disintegrating the gel in an adjacent extracellular fluid over a time period and so that the gel is cleared from the cavity by an elimination pathway.

10. An antifungal formulation comprising a therapeutically effective amount of an antifungal drug dispersed or solubilized in a carrier, wherein the carrier comprises a poloxamer in an amount such that the formulation is the form of a gel at the mammal body temperature and in the form of a liquid at a temperature lower than the mammal body temperature.

11. A method of delivering a drug formulation to a bodily cavity in a mammal comprising:
providing formulation comprising a therapeutically effective amount of an active drug dispersed or solubilized in a carrier, wherein the carrier comprises a poloxamer in an amount such that the formulation is the form of a gel at the mammal body temperature and in the form of a liquid at a temperature lower than the mammal body temperature; and
forming a barrier against pathogen entry into a cavity in an animal by inserting the formulation into a cavity of the animal, whereupon when the formulation becomes warmer by heat from the animal the formulation gels to form an implant and adheres to tissue around a periphery of the cavity to form the barrier.

12. The method of claim 11, further comprising eluting the drug from the gel over a desired time period.

13. The method of claim 11, further comprising eluting the drug from the gel at a desired rate.

14. The method of claim 11, further comprising adhering the gel to an affected tissue to deliver the drug directly to the affected tissue.

15. The method of claim 11, wherein the drug is a veterinary drug.

16. The method of claim 11, wherein the drug comprises an anti-inflammatory drug and an antifungal drug, and the method further comprises treating, preventing or alleviating an allergy over a time period.

17. The method of claim 11, wherein the gel is biodegradable, and the method further comprising dissolving or disintegrating the gel in an adjacent extracellular fluid over a time period and so that the gel is cleared from the cavity by an elimination pathway.

18. A veterinary formulation comprising a therapeutically effective amount of an active veterinary drug dispersed or solubilized in a carrier, wherein the carrier comprises a poloxamer in an amount such that the formulation is the form of a gel at the mammal body temperature and in the form of a liquid at a temperature lower than the mammal body temperature.
